(19) United States
(12) Patent Application Publication
(10) Pub. No.: US 2003/0194446 A1
(43) Pub. Date: Oct. 16, 2003

(54) Zinc oxide compositions for dermatheraputics

(76) Inventors: Lindy K. Akes, Espanola, NM (US); Michael E. Cournoyer, Los Alamos, NM (US)

Correspondence Address: CAHN & SAMUELS LLP, 2000 P STREET NW, SUITE 200, WASHINGTON, DC, 20036, US

(21) Appl. No.: 10/119,304

(22) Filed: Apr. 10, 2002

(51) Int. Cl. A61K 31/7048; A61K 31/704; A61K 31/573; A61K 31/496; A61K 33/22; A61K 31/47; A61K 31/4196

(52) U.S. Cl. 424/642; 514/37; 514/28; 514/171; 514/254.07; 514/312; 514/383

(57) ABSTRACT

20% Zinc oxide formulations including up to 5% of at least one pharmacologically active agent and associated topical methods for improved therapeutic treatment of adverse atopic dermatological conditions.

### I. FIELD OF THE INVENTION

This invention relates to new therapeutic compositions for topical treatment of adverse cutaneous pathologies and dermatitis and more particularly compositions for topical application of creams comprising 20% zinc oxide and an active medicament specific to a targeted condition. The invention recognizes that in combination with other therapeutically active agent(s), 20% zinc oxide in a topically applicable form, e.g. cream or lotion, serves to synergistically enhance the effectiveness of the agent in the treatment of undesirable cutaneous pathologies and dermatological conditions such as psoriasis, eczema, puritus, seborrheic dermatitis, acne, rus rashes, and other inflammatory skin condition.

### II. BACKGROUND OF THE INVENTION

Topical formulations for therapeutic treatment of dermatitis are well known. The inclusion of zinc oxide in such formulations is also well known. Topical zinc oxide-containing creams and lotions are used for a wide variety of purposes ranging from sun-blocks to various ointments.

Topical antiseptics/astringents composed of 20% zinc oxide in a petrolatum, mineral oil, and lanolin ointment base are used in the veterinary field in treatment of superficial wounds of farm animals.

In treatment of humans, topical therapeutic creams/ointments are most commonly used in the treatment of adverse pathologies. Such compositions are used for, for example, psoriasis a hyperproliferative disease of the skin often indicated by red, scalely skin lesions. It is known to use topical steroid containing treatments (e.g. the adrenocortical steroids) for providing temporary remission of the symptoms. It is known to topically treat eczema, another common dermatological malady, with corticosteroids (e.g., 2%-5% hydrocortisone, betamethasone dipropionate, or clobetasol propionate. Acne, involving comedongenic and/or papulopustular lesions, is another commonly topically treated dermatitis.

Zinc oxide is an amphoteric metal oxide commonly used for topical dermal application. Topically applied zinc oxide is typically inert and only slightly bioavailable. When combined with other neutral ingredients, zinc-oxide based lotions do not irritate normal skin at a pH of 4.2 to 5. Two well-known uses of zinc-oxide containing creams are as a sun-block and a skin barrier against irritation. Due to its perceived inactive role, zinc-oxide is used in wide spectrum of topical creams, lotions, and ointments but is not viewed as contributing to the therapeutic efficacy of topically applied pharmacologically active agents.

Topical effectiveness of a pharmaceutically active compound depends on two principal factors; 1) bioavailability of the active ingredient as formulated and 2) the degree of percutaneous absorption, penetration and distribution of the active ingredient to the target site in the skin.

### IV. BRIEF DESCRIPTION OF THE PRIOR ART

Examples of therapeutic uses of zinc oxide include as a counterirritant for cutaneous conditions, an antiseptic and astringent.

Examples of zinc oxide containing formulations include De Gregorio U.S. Pat. No. 6,342,255, which describes a therapeutic preparation for atopic dermatitis combining a select pollen extract in a 10% zinc oxide cream.

Harendza-Harinxma, U.S. Pat. No. 4,847,283, discloses ointments comprising indole derivatives to combat inflammation resulting from Herpes. The formulations include 40% by weight zinc oxide, which may be pre-formulated in the form of DESITIN® brand ointment or with admixtures including petrolatum, lanolin, talc, and cod-liver oil or ethanol or propylene glycol with petrolatum.

In U.S. Pat. No. 5,091,192, Enjolras discloses a diaper rash cream composed of an anti-enzyme such as a chelating agent of 18-23% zinc oxide and titanium oxide in combination and a number of excipients, in a carrier having a balance of water.

Billia et al describe an anti-sun cream in U.S. Pat. No. 5,486,353 for treatment of dermatitis solaris comprising a deprotinated hemodyalysate of mammalian blood combined in some examples, with 10-20% zinc oxide and cosmetic auxiliaries.

Lacefield et al in U.S. Pat. No. 4,021,553, disclose a protective topical anti-inflammatory composition for treatment of conditions such as contact dermatitis, nummular eczema, psoriasis etc., where the ointment includes 20% zinc oxide with 3% triazine in an oil-wax-petrolatum base.

Another composition useful for topical treatment in case of amputation or psoriasis is discussed in Inwood, U.S. Pat. No. 4,512,978. The patent discloses a cream/paste including zinc oxide (optimally 1-3% but mentions up to 20%) which is combined with active ingredients such as urea, coal tar, castor oil, and corn starch.

Other than compounding zinc oxide to be used as an essentially inert element, surprisingly, as evidenced from the foregoing examples, formulations enhancing the effectiveness of topically applied, therapeutically active, dermatological medications have not been developed.

### V. SUMMARY OF THE INVENTION

This invention is directed to novel compositions and associated topical treatment methods employing a therapeutically effective combination of a 20% zinc oxide in dermatitis ameliorating formulations.

An object of this invention is to provide an improved treatment for dermatological pathologies.

Another object of this invention is to provide improved compositions and methods for topical treatment of common skin problems such as atopic dermatitis.

A further object of this invention is to obtain topically applied therapeutics with known bioactive compounds and a 20% zinc oxide based cream/lotion.

Another object of this invention is to enhance the effectiveness of known topically applied, bioactive agents for improving adverse dermatological pathologies by combining such agent(s) in a 20% zinc oxide formulation.

Still a further object of this invention is to provide stable, non-prescription formulations exhibiting improved bio-absorption and duration, thereby minimizing the need for frequent applications.

These and other objects are satisfied by a topical therapueutic composition, comprising: 20% zinc oxide, up to 5% of a therapueutucally active agent selected from the group consisting of adrenocortical steroids such as hydrocortisone, antibiotic mixtures such as Neosporin® a terniary antibiotic formulation including Polymyxin B Sulfate, Bacitracin Zinc and Neomycin, Quinoline, Betamethasone dipropionate, and Ketoconazole, and the balance consisting of a base comprising a mixture of at least two compounds selected from the group consisting of petrolatum, wax, mineral oil, and lanolin.

The foregoing and other objects are satisfied by a topical dermatological regime comprising the steps of atopic dermatological pathologies using the above-described composition comprising the step of applying the composition to the affected area and the surrounding epidermis.

In short, the present invention recognizes the specific synergy associated with topically applied compositions containing known bioactive agents compounded with 20% zinc oxide. The invention contemplates direct cutaneous application on affected target tissue as a method for ameliorating adverse dermatological pathologies.

For definitional purposes and as applicable, as used herein “combining”, “compounded” and like words include any known technique for producing a substantially uniform, topically applicable therapeutic composition and, unless specified, such words are intended to embrace any topical therapeutic formulation process subject to the limitations of the invention.

As used herein “substantially,” “generally,” and other words of degree are relative modifiers intended to indicate permissible variation from the characteristic so modified. It is not intended to be limited to the absolute value or characteristic which it modifies but rather possessing more of the physical or functional characteristic than its opposite, and preferably, approaching or approximating such a physical or functional characteristic.

The following description is shown by way of illustration to the specific embodiments in which the invention may be practiced. The following embodiments are described in sufficient detail to enable those skilled in the art to practice the invention. It is to be understood that other embodiments may be utilized and that changes based on presently known computation chemical and/or functional equivalents may be made without departing from the scope of the invention.

Given the following detailed description, it should become apparent to the person having ordinary skill in the art that the invention herein provides improved, topically applied, dermatological treatments containing bioactive agents in combination with 20% zinc oxide.

Further features and advantages of the present invention, as well as the formulations and treatment regimes, are described in detail below. Given the following enabling description, the invention should become evident to a person of ordinary skill in the art.

### VI. DETAILED DESCRIPTION OF THE INVENTION

The novel compositions for therapeutic treatment of a variety of cutaneous pathologies including atopic dermatitis comprise formulations incorporating 20% Zinc oxide. We have determined that for topical applications, a combination of 20% zinc oxide with one or more active therapeutic agents, in a petrolatum, mineral oil and wax base, provides enhanced therapeutic effectiveness, augments bio-absorption and reduces the frequency of applications required to achieve a substantially equivalent therapeutic effect from compositions not incorporating 20% zinc oxide. Use of the invention also has been observed to increase healing rates and reduce scarring.

The following Table provide specific examples of therapeutic preparations of the invention:

TABLE 1

| | Active | | |
|---|---|---|---|
| Formula | Ingredient | Base | Indications |
| 1 | 1% | Zinc Oxide (20%), | Atopic Dermatitis, |
| | Hydrocortisone | light mineral oil | Psoriasis, Skin |
| | cream | and white | Ulcerations, External |
| | | petrolatum and | Anal Inflammation and |
| | | white wax base. | Irritation, Hemorrhoids |
| 2 | 1% | Zinc Oxide (20%), | Atopic Dermatitis, |
| | Hydrocortisone | lanolin, light | Psoriasis, Skin |
| | cream | mineral oil and | Ulcerations, External |
| | | white petrolatum | Anal Inflammation and |
| | | and white wax base. | Irritation, Hemorrhoids |
| 3 | 5% | Zinc Oxide (20%), | Atopic Dermatitis, |
| | Hydrocortisone | light mineral oil | Psoriasis, Skin |
| | cream | and white | Ulcerations, External |
| | | petrolatum and | Anal Inflammation and |
| | | white wax base. | Irritation, Hemorrhoids |
| 4 | 5% | Zinc Oxide (20%), | Atopic Dermatitis, |
| | Hydrocortisone | lanolin, light | Psoriasis, Skin |
| | cream | mineral oil and | Ulcerations, External |
| | | white petrolatum | Anal Inflammation and |
| | | and white wax base. | Irritation, Hemorrhoids |
| 5 | 1% Neosporin | Zinc Oxide (20%), | Gram Positive Infectious |
| | Base | light mineral oil | Dermatitis, Psoriasis, |
| | | and white | Skin Ulcerations |
| | | petrolatum and | |
| | | white wax base. | |
| 6 | 1% Neosporin | Zinc Oxide (20%), | Gram Positive Infectious |
| | Base | lanolin, light | Dermatitis, Psoriasis, |
| | | mineral oil and | Skin Ulcerations |
| | | white petrolatum | |
| | | and white wax base. | |
| 7 | 5% Neosporin | Zinc Oxide (20%), | Gram Positive Infectious |
| | Base | light mineral oil | Dermatitis,Psoriasis, |
| | | and white | Skin Ulcerations |
| | | petrolatum and | |
| | | white wax base. | |
| 8 | 5% Neosporin | Zinc Oxide (20%), | Gram Positive Infectious |
| | Base | lanolin, light | Dermatitis, Psoriasis, |
| | | mineral oil and | Skin Ulcerations |
| | | white petrolatum | |
| | | and white wax base. | |
| 9 | 1% Neosporin | Zinc Oxide (20%), | Gram Positive/Negative |
| | Base + | light mineral oil | Infectious Dermatitis, |
| | 1% Quinoline | and white | Psoriasis, Skin |
| | | petrolatum | Ulcerations |
| | | and white wax base. | |
| 10 | 1% Neosporin | Zinc Oxide (20%), | Gram Positive/Negative |
| | Base + | lanolin, light | Infectious Dermatitis, |
| | 1% Quinoline | mineral oil and | Psoriasis, Skin |
| | | white petrolatum | Ulcerations |
| | | and white wax base. | |
| 11 | 5% Neosporin | Zinc Oxide (20%), | Gram Positive/Negative |
| | Base + | light mineral oil | Infectious Dermatitis, |
| | 5% Quinoline | and white | Psoriasis, Skin |
| | | petrolatum | Ulcerations |
| | | and white wax base. | |
| 12 | 5% Neosporin | Zinc Oxide (20%), | Gram Positive/Negative |
| | Base + | lanolin, light | Infectious Dermatitis, |
| | 1% Quinoline | mineral oil and | Psoriasis, Skin |
| | | white petrolatum | Ulcerations |
| | | and white wax base. | |
| 13 | 0.05% | Zinc Oxide (20%), | Severe or Complicated |
| | Betamethasone | light mineral oil | Atopic Dermatitis, |
| | Dipropionate | and white | Psoriasis, Skin |
| | | petrolatum | Ulcerations |
| | | and white wax base. | |
| 14 | 0.05% | Zinc Oxide (20%), | Severe or Complicated |
| | Betamethasone | lanolin, light | Atopic Dermatitis, |
| | Dipropionate | mineral oil and | Psoriasis, Skin |
| | | white petrolatum | Ulcerations |
| | | and white wax base. | |
| 15 | 0.025% | Zinc Oxide (20%), | Severe or Complicated |
| | Betamethasone | light mineral oil | Atopic Dermatitis, |
| | Dipropionate | and white | Psoriasis, Skin |
| | | petrolatum | Ulcerations |
| | | and white wax base. | |
| 16 | 0.025% | Zinc Oxide (20%), | Severe or Complicated |
| | Betamethasone | lanolin, light | Atopic Dermatitis, |
| | Dipropionate | mineral oil and | Psoriasis, Skin |
| | | white petrolatum | Ulcerations |
| | | and white wax base. | |
| 17 | 2.0% | Zinc Oxide (20%), | Fungal Dermatitis, |
| | Ketoconazole | light mineral oil | Psoriasis, Skin |
| | | and white | Ulcerations |
| | | petrolatum | |
| | | and white wax base. | |
| 18 | 2.0% | Zinc Oxide (20%), | Fungal Dermatitis, |
| | Ketoconazole | lanolin, light | Psoriasis, Skin |
| | | mineral oil and | Ulcerations |
| | | white petrolatum | |
| | | and white wax base. | |
| 19 | 5.0% | Zinc Oxide (20%), | Fungal Dermatitis, |
| | Ketoconazole | light mineral oil | Psoriasis, Skin |
| | | and white | Ulcerations |
| | | petrolatum | |
| | | and white wax base. | |
| 20 | 5.0% | Zinc Oxide (20%), | Fungal Dermatitis, |
| | Ketoconazole | lanolin, light | Psoriasis, Skin |
| | | mineral oil and | Ulcerations |
| | | white petrolatum | |
| | | and white wax base. | |
| 21 | 1.0% | Zinc Oxide (20%), | Fungal Dermatitis, |
| | Clotrimazole | light mineral oil | Psoriasis, Skin |
| | | and white | Ulcerations |
| | | petrolatum | |
| | | and white wax base. | |
| 22 | 1.0% | Zinc Oxide (20%), | Fungal Dermatitis, |
| | Clotrimazole | lanolin, light | Psoriasis, Skin |
| | | mineral oil and | Ulcerations |
| | | white petrolatum | |
| | | and white wax base. | |

To prepare one of the above-identified compositions in the form of a stable lotion, cream or ointment, the zinc oxide is mixed in a conventional manner with a pharmacologically suitable cream or ointment vehicle/base such as the above-described hydrophilic petrolatum formulas. The active agent, typically available in non-prescription form at the specified concentrations, is mixed thoroughly by agitation with suitable mixing equipment, e.g., a Banbury mixer to create a product with the active agent and the zinc oxide uniformly distributed throughout.

The treatment method using the 20% zinc oxide formulations of the invention has been shown to promote increased bio-absorption of the active agent and increased the clinical responsiveness to correspondingly decrease the frequency of applications. Additionally, observation of persons using the compositions and treatment regimes of the invention exhibited more rapid healing and reduced the overall number of doctor visits dedicated to treatment therapy for the adverse dermatological pathology. Consequently, treatment according to the invention, reduces overall costs.

While various embodiments of the present invention have been described above, it should be understood that they have been presented by way of example only, and not limitation. Thus, the breadth and scope of the present invention should not be limited by any of the above-described exemplary embodiments, but should be defined only in accordance with the following claims and their equivalents.

We claim:

1. A topical therapueutic composition, comprising:
20% zinc oxide;
0.025-5% of a therapueutucally active agent selected from the group consisting of adrenocortical steroids, Polymyxin B Sulfate, Bacitracin Zinc and Neomycin, Quinoline, Betamethasone dipropionate, Pramoxime hydrochloride, Clotrimazole, and Ketoconazole; and
a base comprising a mixture of at least two compounds selected from the group consisting of petrolatum, wax, mineral oil, and lanolin.

2. The composition of claim 1 where the adrenocortical steroids are selected from the group consisting of hydrocortisone and hydrocortisone acetate.

3. The composition of claim 1 with 1% Quinoline.

4. The composition of claim 3 with 1% Neosporin.

5. The composition of claim 1 with 5% Quinoline.

6. The composition of claim 5 with 1% Neosporin.

7. The composition of claim 1 with 1% Pramoxime hydrochloride.

8. The composition of claim 1 with 2% Ketoconazole.

9. The composition of claim 1 with 1% Clotrimazole.

10. The composition of claim 1 with 0.025% Betamethasone dipropionate.

11. The composition of claim 1 with 0.05% Betamethasone dipropionate.

12. The composition of claim 1 where the base comprises of white petrolatum.

13. The composition of claim 1 where the base comprises of white wax.

14. The composition of claim 1 where the base comprises of light mineral oil.

15. The method of treating atopic dermatological pathologies using the composition of claim 1 comprising the step of applying the composition to the affected area and the surrounding epidermis.
